(19) United States
(12) Patent Application Publication
(10) Pub. No.: US 2014/0024931 A1
(43) Pub. Date: Jan. 23, 2014

(54) Data Encoders for Medical Devices and Related Methods

(71) Applicants: David Winston, Winchester, MA (US); Paul Murray, Groton, MA (US); Victor Grinberg, Somerville, MA (US); Marc Davidson, Andover, MA (US)

(72) Inventors: David Winston, Winchester, MA (US); Paul Murray, Groton, MA (US); Victor Grinberg, Somerville, MA (US); Marc Davidson, Andover, MA (US)

(73) Assignee: LIGHTLAB IMAGING, INC., Westford, MA (US)

(21) Appl. No.: 13/554,596

(22) Filed: Jul. 20, 2012

(51) Int. Cl. A61B 6/00; A61B 8/12; A61B 6/02; A61B 5/00; A61B 1/07

(52) U.S. Cl. 600/427; 600/300; 600/478; 600/467; 600/407

(57) ABSTRACT

In part, the invention relates to systems, methods, and devices that store and retrieve information associated with an imaging probe such as an ultrasound probe, an optical coherence tomography probe, a multimodal probe and other probes. The information that is stored relates to one or more measurable properties for a specific imaging probe. Thus, when each probe is manufactured there can be variations in its length, brightness, angular alignment of its constituent elements, and various other probe specific measurements. In turn, these measurements can be used to calibrate or otherwise use a given probe with a system that collects, stores, or otherwise processes information collected using each probe.

### BACKGROUND

Various imaging modalities such as optical coherence tomography, intravascular ultrasound, and others collect data with respect to a sample using a probe. The probe and the associated system that processes the signals it collects often require calibration to operate or otherwise reduce noise in the resultant image or other information derived from the collected signals. Given the variation in manufacturing steps and the number of interconnected optical, electrical, and mechanical systems that are used in various image data collection systems, it is not surprising that calibrating such systems or components thereof is challenging. In addition, other medical devices have operational parameters or other features that can vary based on device specific measurements and characteristics.

Accordingly, there is a need for fast and cost-effective calibration techniques, medical device control methods, and related devices for use with various probes, devices, control systems and imaging systems. Embodiments of the invention address these needs and others.

### SUMMARY

In part, the invention relates to systems, medical devices, data encoders and methods that encode manufacturing parameters or other parameters. In one embodiment, these parameters are measured on a per component basis for one or more components or subsystems of a data collection probe such as an optical coherence tomography (OCT) probe, intravascular ultrasound (IVUS) probe, a combination OCT and IVUS probe and others. The measured parameters are stored using a data encoding encoder or data encoding element. The data encoder can be implemented using a radio-frequency identification (RFID) chip or other wireless technology, a barcode, or other active or passive means. In one embodiment, the data encoder is implemented using a near field communication (NFC) device such as a NFC chip or other device. The data encoder can be powered or unpowered in some embodiments.

In one embodiment, the data encoder is a tag responsive to an electromagnetic signal. The data encoder can be configured to include one or more data sets in a suitable machine readable format. A first data set can include parameters relating to an acoustic wave-based data collection element. Alternatively, a first data set can include parameters relating to an interferometry-based data collection element such as one or more components of an OCT probe.

In one embodiment, an image data collection probe comprises one or more sheaths with one or more data collection elements disposed therein. One or more of the data collection elements are rotatable in one embodiment. They can rotate separately or together as provided for in a given embodiment. The data encoder or tag encoding the relevant data can be configured to be tamper resistant such as by embedding some or all of the data encoder within one of the sheaths or by applying a coating or layer thereupon such a data encoder.

In addition, the data encoder can be configured to transmit, be read, or otherwise make the one or more parameters available to a circuit or processor-based system. Once received by the system, the probe-specific parameters can be used to expedite calibration of the image data collection system prior to collecting data with such a probe or otherwise increase the signal to noise ratio for a given process or stage of data collection or processing. Improving the setup time and reducing noise in the data collected are some advantages of this approach.

In one aspect, the invention relates to an apparatus. The apparatus includes a medical device comprising a data collection subsystem; the medical device having a quantitative parameter, wherein the quantitative parameter is a measured value; and a data encoder configured to encode the quantitative parameter in a machine readable format, the data encoder attached to the medical device.

In one embodiment, the data encoder is a radio-frequency identification device. In one embodiment, the medical device is an image data collection probe and the data collection subsystem comprises an optical fiber. In one embodiment, the quantitative parameter is a length measurement of the optical fiber or a value derived therefrom. In one embodiment, the quantitative parameter is an intensity profile of tight measured with respect to the optical fiber. In one embodiment, the quantitative parameter is an angular alignment of the optical fiber.

In one embodiment, the medical device is a combination OCT and IVUS probe and the data collection subsystem comprises an ultrasound transducer and an optical element. In one embodiment, the quantitative parameter comprises one more measurements configured to specify an offset between the ultrasound transducer and the optical element. In one embodiment, the optical element is selected from the group consisting of the optical fiber, a GRIN lens, abeam director, a beam forming end face of the optical fiber and an angled surface in optical communication with an interferometer. In one embodiment, the medical device is an image data collection probe and the data collection subsystem comprises an ultrasound transducer and wherein the quantitative parameter is a physical characteristic of the ultrasound transducer.

In one embodiment, the physical characteristic is an acoustic efficiency, one or more dimensions of the ultrasound transducer, and an ultrasound transducer drive voltage. In one embodiment, the apparatus further includes a reader configured to receive the quantitative parameter from the data encoder. In one embodiment, the apparatus further includes a control system comprising a processor configured to execute a software-based method using the quantitative parameter received by the reader, the control system in communication with the reader. In one embodiment, wherein executing the software-based method using the quantitative parameter is faster or otherwise has a signal to noise ratio improvement with respect to a resultant image relative to executing the software-based method without having the quantitative parameter. In one embodiment, wherein the software-based method changes an optical path length dimension used by the control system in response to optical path length data encoded as the quantitative parameter and measured with respect to the image data collection probe.

In one aspect, the method of collecting image data includes receiving a quantitative parameter from a data encoder, the quantitative parameter derived from or comprising a measured value of a disposable data collection probe component; compensating for variations in disposable data collection probes using the quantitative parameter as an input to a control system; and collecting image data using the disposable data collection probe in response to one or more control signals generated at least partially based upon the quantitative parameter.

In one embodiment, the disposable data collection probe component is selected from the group consisting of an optical fiber, an ultrasound transducer, a GRIN lens, abeam director, a sheath, electrical leads, a catheter, and a flush solution. In one embodiment, the step of compensating for variations further includes the step of operating within an acoustic efficiency range at least partially provided by an acoustic efficiency encoded as the quantitative parameter.

In one embodiment, the step of compensating for variations further includes the step of tracking relative positions of an acoustic beam and an optical beam using one or offset measurements encoded as the quantitative parameter. In one embodiment, the method of claim further includes the step of increasing a signal to noise ratio in an image formed using the image data by adjusting for a path imbalance due to a deviation from a predetermined optical fiber length and the optical fiber length measured with respect to the disposable data collection probe. In one embodiment, the quantitative parameter is configured to identify a data collection probe.

This Summary is provided merely to introduce certain concepts and not to identify any key or essential features of the claimed subject matter.

### BRIEF DESCRIPTION OF DRAWINGS

The figures are not necessarily to scale, emphasis instead generally being placed upon illustrative principles. The figures are to be considered illustrative in all aspects and are not intended to limit the invention, the scope of which is defined only by the claims.

FIG. 1 is a schematic diagram that shows an imaging probe and a related system suitable for using encoded information in accordance with an illustrative embodiment of the invention.

FIG. 2 is a schematic diagram that shows a medical device and a related system suitable for using encoded information in accordance with an illustrative embodiment of the invention.

FIG. 3 is a schematic diagram that shows a plurality of optical fiber portions of varying lengths suitable for use in an imaging probe in accordance with an illustrative embodiment of the invention.

FIGS. 4A and 4B area schematic diagrams of imaging probe cross-sections that show angular position variations that can be measured and encoded in accordance with an illustrative embodiment of the invention.

FIG. 4C is a schematic diagram of a multimodal data collection probe such as a combination intravascular ultrasound and optical coherence tomography data collection probe having a data encoder in accordance with an illustrative embodiment of the invention.

FIGS. 5A and 5B are flow diagrams that show a data collection system related method configured to use information from a data encoder in accordance with an illustrative embodiment of the invention.

### DETAILED DESCRIPTION

There are various categories of medical devices that can be used to perform procedures, collect data, and be delivered for use or installation in a patient. In various circumstances, the use of the medical device requires a calibration or other modification step that can use one or more quantitative parameters as input for that step or as a threshold or control parameter.

Imaging systems are one category of medical devices that can so use a quantitative parameter such as a measured value of a given device. Optical coherence tomography (OCT) systems, intravascular ultrasound (IVUS) systems and systems which combine elements of OCT and IVUS are examples of such imaging systems. These systems are complex and can include several components. These components can include optical fiber-based probes, beam directors, GRIN lenses, ultrasound transducers such as piezoelectric stacks, catheters, control and data collection systems, and various other components. When a given probe is manufactured, there can be variations in the length of the optical fiber, the optical properties of any individual component of the optical train, alignment variations associated with the ultrasound transducer relative to the other probe components, angular alignment variation, and other imaging probe specific characteristics or parameters.

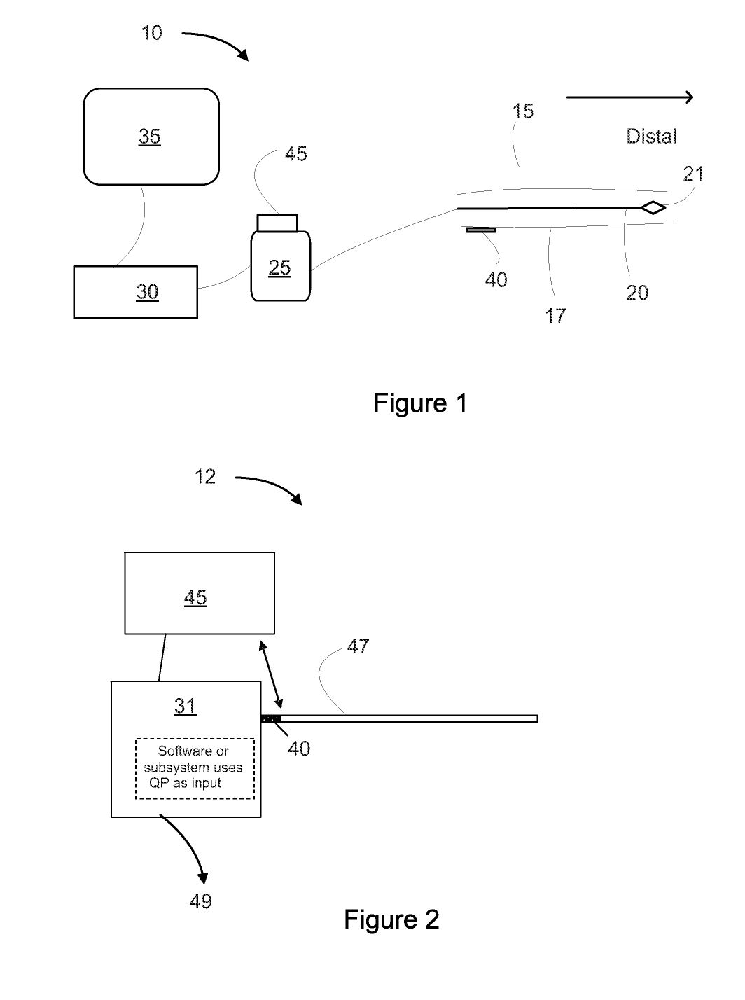

In part, the invention relates to systems, methods and related devices that encode medical device information, such as for example imaging probe specific characteristics or parameters, on a per medical device basis. Exemplary embodiments of two such systems are shown in FIGS. 1 and 2. In FIG. 1, the system 10 includes various components relating to an image data collection system. The system 12 of FIG. 2 includes a more general system suitable for use with various medical devices. Either systems 10, 12 can be used with a data collection probe or other medical device.

In FIG. 1, a probe 15 that includes a catheter 17 and an optical fiber 20 is depicted. As shown, the probe is configured to be introduced into a lumen, such as a blood vessel, and moved distally into the lumen. The optical fiber 20 can be used to transmit light and collect light from a blood vessel or other sample for processing by an optical coherence tomography system. A beam director 21 can be in optical communication with the fiber 20. The probe 15 can include any type of probe, whether or not used for imaging, which has parameters which vary on a per probe basis. The probe 15 can be pulled back as the fiber 20 and beam director 21 rotate to collect image data.

The image data collection system can include an interface unit 25 that can include a rotary coupler (not shown) that connects to the optical fiber 20. The interface unit can, in turn, be in electrical or optical communication with an OCT system 30. This system 30 can include an interferometer, photoreceiver, a computer, clocking circuits, or other components, either alone or in combination. The OCT system can be in electrical communication with a display or other output device to show images generated using the image data collected by the probe 15. This image data can include a three-dimensional tomographic view or cross-sections of the lumen that was scanned using the probe 15.

As shown, a data encoder or data encoding element 40 is adjacent to, connected to or integrated as part of the probe 15. The data encoder can be disposed in a sheath that forms part of the data collection probe to prevent tampering. This data encoder 40 can be configured to encode probe specific quantitative or measured parameters that are obtained when the probe is assembled. A decoder 45 such as a reader, scanner, wireless device, or any other device suitable for decoding or otherwise receiving the information from the data encoder 40 can be used. The decoder 45 is in communication with OCT system 30 or another device that relays the quantitative parameter(s). Although the decoder 45 is shown disposed relative to the interface unit 25 this configuration is provided as an example. The decoder can be positioned at any suitable location such that it can received information from the encoder 40.

A quantitative parameter (QP) or multiple QPs can be are stored or encoded a using a data encoding element 45. This element or encoder 45 can include a RFID chip, symbol, etching, mechanical element, machine readable element, wireless device or other technology that delivers, transmits, reads, encodes, stores or otherwise makes the probe specific measurements available for use my an electronic, optical, or mechanical system. For example, one or more parameters that are probe specific, such as its length, can be automatically input to a control system or decoded and then transmitted for use as an input for one or more computer programs, circuits, or other methods. These programs, methods, or circuits can use the decoded QP to expedite calibration or initialization of the OCT system 30 or another subsystem of systems 10, 12 prior to using such a probe.

The QP can also be used to specify other actions or cause them to occur automatically. For example, a stent may have encoded information that encodes the clamping pressure to apply prior to its installation. A QP can also be used to set safety thresholds such as maximum spin rates and other parameters. In one embodiment, for a given image data collection system, different probes/catheters can be used. Accordingly, in one embodiment the QP can include the probe/catheter type. In FIG. 2, a medical device 47 has an associated data encoder 40 having an encoded QP. The encoder 40 can be decoded or otherwise interacted with using the decoder 45 so that the QP is available for use. As shown, the QP can be used by a software program, control system, graphic user interface, or other device or subsystem 31 as applicable to the field the medical device 47 relates. Wireless signaling is preferred in one embodiment such that the QP encoded in the encoder is readable by a suitable decoder or other device without direct contact. This can be achieved by receiving an electromagnetic signal or detecting a property of the encoder. An RFID system is one example of such a wireless or contactless embodiment.

In one embodiment, the QPs are used to encode manufacturing details or specific operating values that are device specific. This is useful for various imaging modalities such as OCT and IVUS. The construction of an imaging probe such as a single imaging mode probe or a multimodal probe is subject to various alignment, brightness, component positioning, and length variations for a given probe.

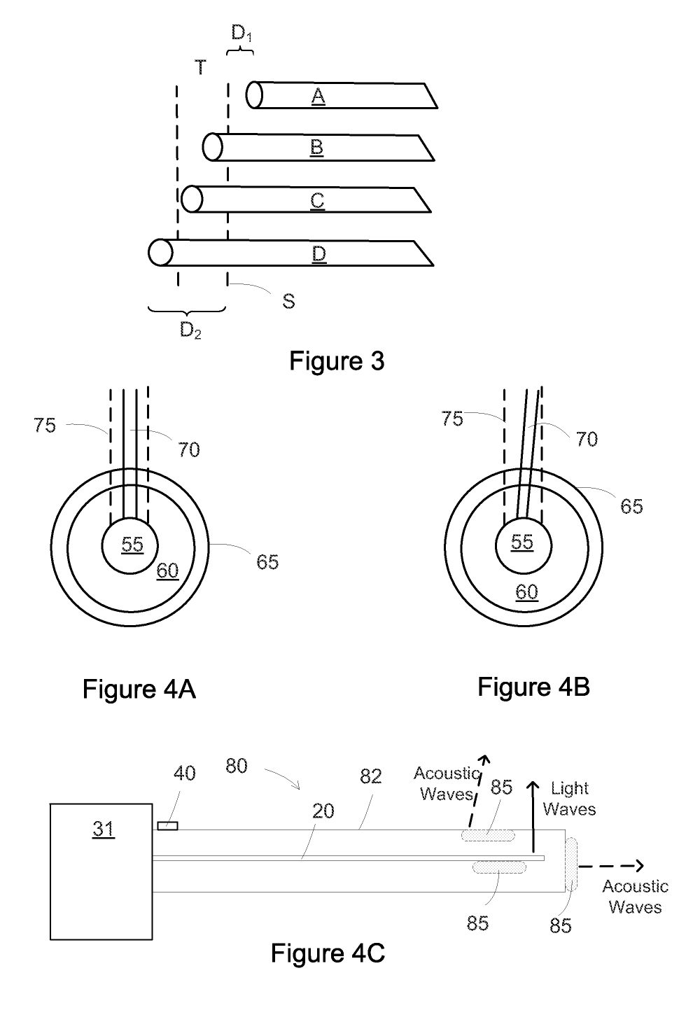

For example, in one embodiment the lengths of the optical fiber portion(s) used to assemble different probes can vary. This is shown by the optical fiber portions A-D in FIG. 3. As part of the process of manufacturing OCT probes, optical fibers can be cut and used as part of the probe. The length of the optical probe sections is likely to vary when the probes are handmade. A tolerance range T can be set that specifies the acceptable length range for certain probes. As shown, optical fibers A and D are outside this range T as having a length that is too short or too long. In some embodiments, all four optical fiber portions A-D can be used by encoding their individual measured lengths as a QP in an encoder element. This encoder element can be associated with each respective imaging probe that will incorporate the fibers A-D by attaching to part of the probe.

This approach can increase overall manufacturing yield by allowing probes to be used that have lengths outside of the tolerance range T. This follows because the varying lengths of each fiber can be encoded and associated with each probe to provide path length details that can be used with other optical path lengths. These other path lengths can include the arms of the interferometer or coils of fiber used as part of the optical source in an OCT system. Thus, in one embodiment a standard optical fiber length can be measured. In turn, a relative length deviation of each optical fiber, used in a data collection probe, can be compared relative to such a standard optical fiber. In turn, such a deviation in length relative to the standard optical fiber can be encoded. Alternatively, the ratio of a fiber length to a standard length or another correlated variable associated with a measured length of an optical fiber, which can include a subset of its overall length, can be encoded as QPs in one embodiment.

The arrangement of optical fiber portions in FIG. 3 can also be evaluated relative to a standard optical fiber. For example, a standard optical fiber can have a length S shown by the right most dotted line. Other fiber portions may deviate in terms of length or other dimensions from those of a standard optical fiber of predetermined dimensions. The length measurements D₁ and D₂ represent deviations in length relative to the standard length S of a standard optical fiber portion. These types of length measurements D₁ and D₂, can be encoded as quantitative parameters in the data encoders. In one embodiment, a quantitative parameter can include a length measurement of the optical fiber or a value derived there from, which can include, without limitation, a deviation from an optical fiber standard, a ratio of a measured optical fiber length and another value, and any other values that include such a length measurement.

FIGS. 4A and 4B are cross-sectional views of an imaging probe. An optical fiber 55 is disposed in a lumen 60 defined by a sheath 65 such as a catheter wall. The optical fiber 55 can include a beam director that is configured to direct a first beam 70, such as a beam of light 70 through the sheath 65. A second beam 75 such as light beam or an ultrasound beam can be generated by a transducer or optical element disposed adjacent to or otherwise relative to the beam director of the optical fiber portion 55. The transducer or optical element configured to direct a beam 75 at the same region as the first beam 70 has an angular alignment and/or offset relative to the beam director that directs the first beam 70. Similarly, the two beams 70, 75 have an angular alignment and/or offset. In FIG. 4A the beams 70, 75 are substantially aligned or parallel. As shown in FIG. 4B, the angular alignment between the elements which generate or otherwise direct the beams 70, 75 are misaligned by an angular measure. This angular measure can be determined and encoded as a QP. Similarly, the relative positions or offset of an optical source, such as an optical fiber, and an acoustic source, such as a transducer, to each other can be determined and encoded as a QP. In turn, this angular measure, and/or offset, and/or deviation can be used to mitigate or reduce the effects of such misalignment in some embodiments of the invention. This can result in signal to noise ratio improvements relative to various image data collection and processing stages.

If QPS are obtained and encoded, the alignment of components of an imaging probe such as the optical beam director and the ultrasound beam generating stack can be allowed to vary such that precise alignment is not required during manufacturing. This can allow for broader manufacturing tolerances and can increase manufacturing yield. Thus, in part, the invention relates to methods and devices that allow a greater margin of error for certain tolerances between parts of the probe or the measurements of a given component.

FIG. 4C is a schematic diagram of a multimodal data collection probe 80 having a sheath 82. The probe 80 is connected to subsystem 31 referenced above as various suitable components with respect to FIG. 2. In one embodiment the length of the optical fiber 20 is measured and encoded as a QP. As shown in dotted lines one or more ultrasound transducers 85 can be positioned relative to the sheath 82 and optical fiber 20 or be connected to a torque wire or other elongate member (not shown). Typically, one transducer 85 is used. In this embodiment, two imaging modes, OCT and IVUS are used. As such, with regard to probe 80 various physical characteristics of the probe can be encoded as QPs. The relative positions or offset of an optical source, such as an optical fiber 20, and an acoustic source, such as a transducer 85, to each other can be determined and encoded as a QP. These can be encoded as coordinates, distances, or in any other suitable format. With respect to this embodiment and others, the QP are received by a subsystem 31 and then transformed using applicable software to control signals that can include processor instructions, clocking signals, and others to adjust or compensate for the optical, acoustic, mechanical, or other physical proprieties of the probe. In general, by using QPs as an input, signal to noise ratios in data processing steps and in images generated using data obtained in response to such control signals is increased. This increase in signal to noise ratio is at least an increase relative to an unadjusted or uncompensated data collection session when no QP is available.

Once the relevant measurements and parameters relating to the probe have been obtained such as the measured offset (misalignment, both rotational and axial), brightness, length, and others, one or more of these parameters can be stored in a data encoding element such as a barcode, glyph, RFID device, or other active or passive wireless devices that can deliver data to an a control or calibration system and or be scanned or otherwise interrogated such that such parameters are extracted. These parameters can be used to authenticate a probe and provide catheter specific information that enables the control system to more accurately and/or more expeditiously use, initialize, or calibrate the image data collection system.

One or more of these parameters can be used as calibration data that is specific to a particular probe. In this way, knowing probe specific measurements and other data allows the system to use specific details rather than averages or guesses relating to a given probe. Then software in the system can make adjustments based on this calibration data. For example, one or more of these parameters can be used to perform a rotational adjustment (such as by offsetting some number of scan lines). These parameters can also be used to perform axial adjustments.

The probe encoded parameters can also be used to expedite the process of calibrating an OCT system by providing one or more initial parameters such as the probe distance from a sheath. By using a OP from an encoder that includes a probe-specific measure, instead of spending processing cycles on a hunting or iterative search algorithm trying to find a sheath position or other data point of interest, that information can be immediately available for the computer to use. Accordingly, any calibration or other software-based methods performed in a reduced time period relative to a system in which the initial parameters are not encoded are embodiments of the invention.

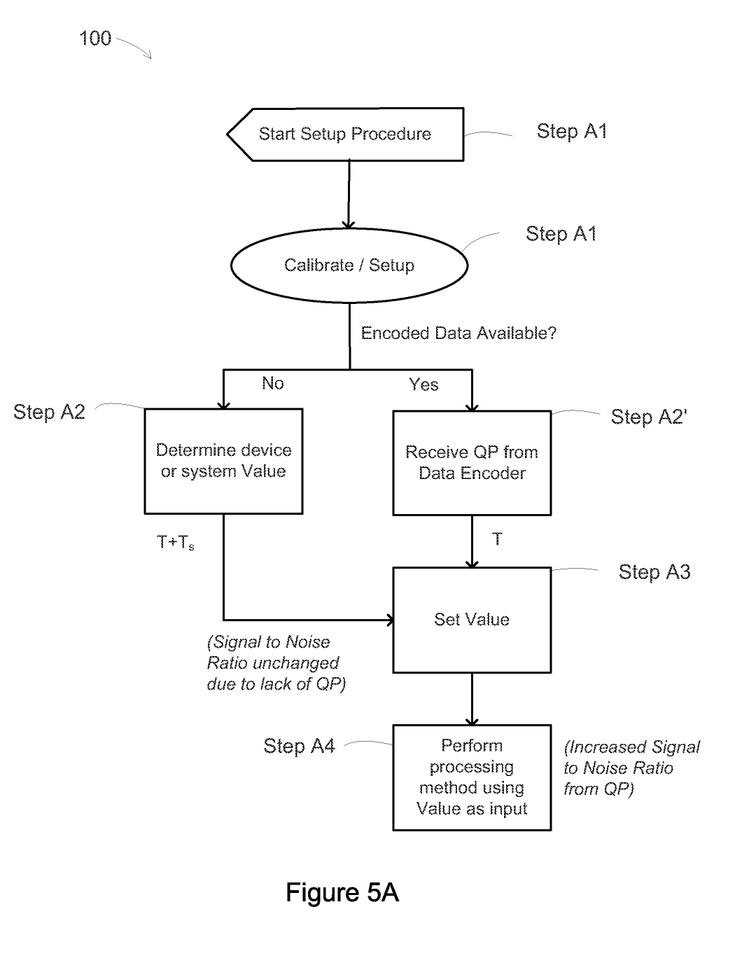

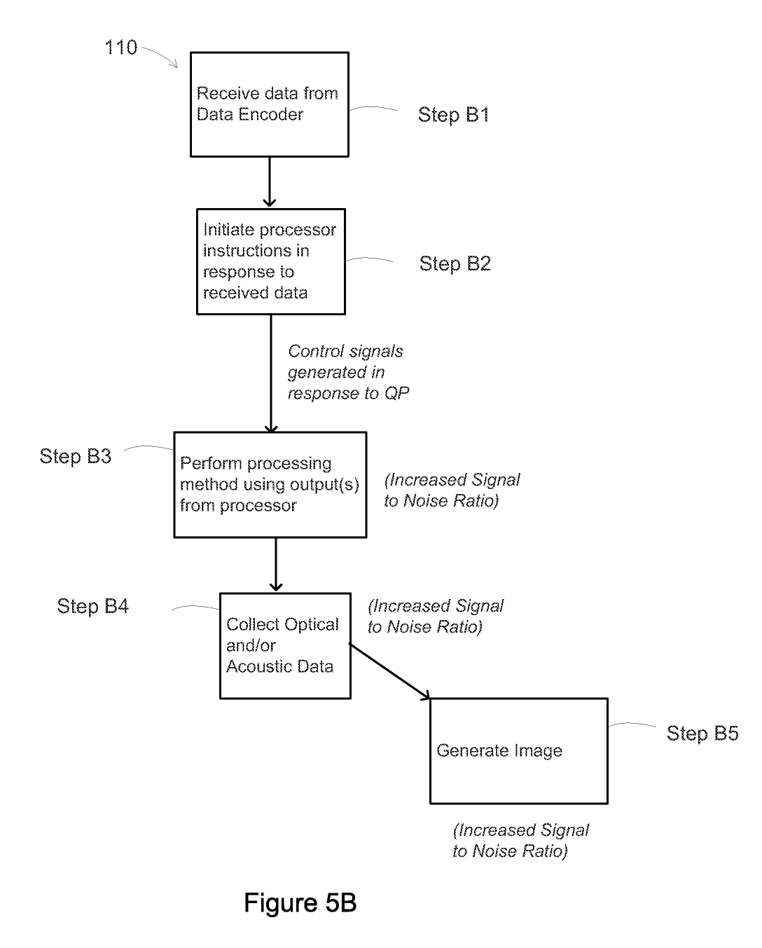

In FIG. 5A, a method 100 suitable for implementing by a processor-based system, circuit or control system that uses QPs as inputs is shown. In one embodiment, this method is suitable for calibrating a data collecting system. Initially, prior to a clinician performing a pullback procedure during which image data is obtained with respect to a lumen of interest, the image data collection system can be configured relative to a disposable imaging probe. Given that each disposable data collection probe may present different optical properties, optical path lengths, acoustic properties as indicated by the examples of FIGS. 3, 4A, 4B, and 4C, a data collection system can be calibrated or setup such that the properties of the disposable probe are used when image data is collected. This can result in improved signal to noise ratios at different stages of data collection, storage, and image generation.

As part of the setup procedure of FIG. 5A, a probe authentication step, calibration method, or other methods can be performed. In this embodiment, additional details are provided with respect to a calibration or initialization method. In some instances the image data collection system, such as an IVUS or OCT or combination IVUS and OCT system, may be used with an imaging probe that lacks a data encoder. In this scenario, the calibration method proceeds for a time T+Ts to find a particular position, such as a position of a sheath that surrounds the optical fiber, offset, or other parameter, and then proceed to calibrate the system using that set position. Alternatively, if a data encoder is present, the relevant position or other QP that is relevant can be read and then set in the computer or otherwise used as an input from which calibration or other actions can be initiated. Reading the encoded QP allows the relevant method to proceed at time resulting in a faster method. The time Ts represents the savings in time associated with the ability to skip steps in the process as a result of the OP being available as opposed to being iteratively determined or iteratively determining a parameter that can be derived or calculated using the QP.

FIG. 5B shows a general method embodiment 110 of the invention in which data encoded in a tag such as an RFID or NFC device can be used as an input to a control system, which can include any of the data processing, clocking, or other controlling components of an image data collection, OCT, IVUS, or combination system. In one embodiment, the method can include the steps of receiving data from a data encoder. The method can also include initiating processor instructions or generating control signals in response to received data from data encoder. A processing, signal generating step or transforming data step can be performed using outputs from the processor which can include control signals or software instructions. With these instructions or signals, the system is configured to collect optical and/or acoustic data with respect to a sample such as a blood vessel. In one embodiment, an image can be generated. At these different stages and steps, the signal to noise ratio of an output or collected data can be increased by making adjustments and/or compensating for variations or other factors associated with using disposable image probes by storing such variations or other related data in a data encoder.

Non-Limiting Software Features and Embodiments for Implementing OCT and/or IVUS Methods and Systems

The following description is intended to provide an overview of device hardware and other operating components suitable for performing the methods of the invention described herein. This description is not intended to limit the applicable environments or the scope of the invention. Similarly, the hardware and other operating components may be suitable as part of the apparatuses described above. The invention can be practiced with other system configurations, including personal computers, multiprocessor systems, microprocessor-based or programmable electronic device, network PCs, minicomputers, mainframe computers, and the like.

Some portions of the detailed description are presented in terms of algorithms and symbolic representations of operations on data bits within a computer memory. These algorithmic descriptions and representations can be used by those skilled in the computer and software related fields. In one embodiment, an algorithm is here, and generally, conceived to be a self-consistent sequence of operations leading to a desired result. The operations performed as methods stops or otherwise described herein are those requiring physical manipulations of physical quantities. Usually, though not necessarily, these quantities take the form of electrical or magnetic signals capable of being stored, transferred, combined, transformed, compared, and otherwise manipulated.

Unless specifically stated otherwise as apparent from the following discussion, it is appreciated that throughout the description, discussions utilizing terms such as “processing” or “computing” or calculating or “comparing, “calibrating” “generating” or “determining” or “displaying” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The present invention, in some embodiments, also relates to apparatus for performing the operations herein. This apparatus may be specially constructed for the required purposes, or it may comprise a general purpose computer selectively activated or reconfigured by a computer program stored in the computer.

The algorithms and displays presented herein are not inherently related to any particular computer or other apparatus. Various general purpose systems may be used with programs in accordance with the teachings herein, or it may prove convenient to construct more specialized apparatus to perform the required method steps. The required structure for a variety of these systems will appear from the description below.

Embodiments of the invention may be embodied in many different forms, including, but in no way limited to, computer program logic for use with a processor (e.g., a microprocessor, microcontroller, digital signal processor, or general purpose computer), programmable logic for use with a programmable logic device, (e.g., a Field Programmable Gate Array (FPGA) or other PLD), discrete components, integrated circuitry (e.g., an Application Specific Integrated Circuit (ASIC)), or any other means including any combination thereof. In a typical embodiment of the present invention, some or all of the processing of the data collected using an OCT probe and the processor-based system is implemented as a set of computer program instructions that is converted into a computer executable form, stored as such in a computer readable medium, and executed by a microprocessor under the control of an operating system. Thus, query response and input data are transformed into processor understandable instructions suitable for generating OCT data, encoding data, decoding data, reading RFID tags, calibrating an OCT system using a QP, performing a medical device specific action based on or in response to a QP, and other features and embodiments described above.

Computer program logic implementing all or part of the functionality previously described herein may be embodied in various forms, including, but in no way limited to, a source code form, a computer executable form, and various intermediate forms (e.g., forms generated by an assembler, compiler, linker, or locator). Source code may include a series of computer program instructions implemented in any of various programming languages (e.g., an object code, an assembly language, or a high-level language such as Fortran, C, C++, JAVA, or HTML) for use with various operating systems or operating environments. The source code may define and use various data structures and communication messages. The source code may be in a computer executable form (e.g., via an interpreter), or the source code may be converted (e.g., via, a translator, assembler, or compiler) into a computer executable form.

The computer program may be fixed in any form (e.g., source code form, computer executable form, or an intermediate form) either permanently or transitorily in a tangible storage medium, such as a semiconductor memory device (e.g., a RAM, ROM, PROM, EEPROM, or Flash-Programmable RAM), a magnetic memory device (e.g., a diskette or fixed disk), an optical memory device (e.g., a CD-ROW, a PC card (e.g., PCMCIA card), or other memory device. The computer program may be fixed in any form in a signal that is transmittable to a computer using any of various communication technologies, including, but in no way limited to, analog technologies, digital technologies, optical technologies, wireless technologies (e.g., Bluetooth), networking technologies, and internetworking technologies. The computer program may be distributed in any form as a removable storage medium with accompanying printed or electronic documentation (e.g., shrink-wrapped software), preloaded with a computer system (e.g., on system ROM or fixed disk), or distributed from a server or electronic bulletin board over the communication system (e.g., the Internet or World Wide Web).

Hardware logic (including programmable logic for use with a programmable logic device) implementing all or part of the functionality previously described herein may be designed using traditional manual methods, or may be designed, captured, simulated, or documented electronically using various tools, such as Computer Aided Design (CAD), a hardware description language (e.g., VHDL or AHDL), or a PLD programming language (e.g., PALASM, ABEL, or CUPL).

Programmable logic may be fixed either permanently or transitorily in a tangible storage medium, such as a semiconductor memory device (e.g., a RAM, ROM, PROM, EEPROM, or Flash-Programmable RAM), a magnetic memory device (e.g., a diskette or fixed disk), an optical memory device (e.g., a CD-ROM), or other memory device. The programmable logic may be fixed in a signal that is transmittable to a computer using any of various communication technologies, including, but in no way limited to, analog technologies, digital technologies, optical technologies, wireless technologies (e.g., Bluetooth), networking technologies, and internetworking technologies. The programmable logic may be distributed as a removable storage medium with accompanying printed or electronic documentation (e.g., shrink-wrapped software), preloaded with a computer system (e.g., on system ROM or fixed disk), or distributed from a server or electronic bulletin board over the communication system (e.g., the Internet or World Wide Web).

Various examples of suitable processing modules are discussed below in more detail. As used herein a module refers to software, hardware, or firmware suitable for performing a specific data processing or data transmission task. Typically, in a preferred embodiment a module refers to a software routine, program, or other memory resident application suitable for receiving, transforming, routing and processing instructions, or various types of data such as measured probe parameters, quantitative parameters, encoding schemes, decoding schemes, calibration data, probe lengths, probe measurements, probe intensity, and other information of interest.

Computers and computer systems described herein may include operatively associated computer-readable media such as memory for storing software applications used in obtaining, processing, storing and/or communicating data. It can be appreciated that such memory can be internal, external, remote or local with respect to its operatively associated computer or computer system.

Memory may also include any means for storing software or other instructions including, for example and without limitation, a hard disk, an optical disk, floppy disk, DVD (digital versatile disc), CD (compact disc), memory stick, flash memory, ROM (read only memory), RAM (random access memory), DRAM (dynamic random access memory), PROM (programmable ROM), EEPROM (extended erasable PROM), and/or other like computer-readable media.

In general, computer-readable memory media applied in association with embodiments of the invention described herein may include any memory medium capable of storing instructions executed by a programmable apparatus. Where applicable, method steps described herein may be embodied or executed as instructions stored on a computer-readable memory medium or memory media. These instructions may be software embodied in various programming languages such as C++, C, Java, and/or a variety of other kinds of software programming languages that may be applied to create instructions in accordance with embodiments of the invention.

The aspects, embodiments, features, and examples of the invention are to be considered illustrative in all respects and are not intended to limit the invention, the scope of which is defined only by the claims. Other embodiments, modifications, and usages will be apparent to those skilled in the art without departing from the spirit and scope of the claimed invention.

The use of headings and sections in the application is not meant to limit the invention; each section can apply to any aspect, embodiment, or feature of the invention.

Throughout the application, where compositions are described as having, including, or comprising specific components, or where processes are described as having, including or comprising specific process steps, it is contemplated that compositions of the present teachings also consist essentially of, or consist of, the recited components, and that the processes of the present teachings also consist essentially of or consist of the recited process steps.

In the application, where an element or component is said to be included in and/or selected from a list of recited elements or components, it should be understood that the element or component can be any one of the recited elements or components and can be selected from a group consisting of two or more of the recited elements or components. Further, it should be understood that elements and/or features of a composition, an apparatus, or a method described herein can be combined in a variety of ways without departing from the spirit and scope of the present teachings, whether explicit or implicit herein.

The use of the terms “include,” “includes,” “including,” “have,” “has,” or “having” should be generally understood as open-ended and non-limiting unless specifically stated otherwise.

The use of the singular herein includes the plural (and vice versa) unless specifically stated otherwise. Moreover, the singular forms “a,” “an,” and “the” include plural forms unless the context clearly dictates otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions may be conducted simultaneously.

Where a range or list of values is provided, each intervening value between the upper and lower limits of that range or list of values is individually contemplated and is encompassed within the invention as if each value were specifically enumerated herein. In addition, smaller ranges between and including the upper and lower limits of a given range are contemplated and encompassed within the invention. The listing of exemplary values or ranges is not a disclaimer of other values or ranges between and including the upper and lower limits of a given range.

What is claimed is:

1. An apparatus comprising
a medical device comprising a data collection subsystem; the medical device having a quantitative parameter, wherein the quantitative parameter is a measured value; and
a data encoder configured to encode the quantitative parameter in a machine readable format, the data encoder attached to the medical device.

2. The apparatus of claim 1 wherein the data encoder is a radio-frequency identification device.

3. The apparatus of claim 2 wherein the medical device is an image data collection probe and the data collection subsystem comprises an optical fiber.

4. The apparatus of claim 3 wherein the quantitative parameter is a length measurement of the optical fiber or a value derived therefrom.

5. The apparatus of claim 3 wherein the quantitative parameter is an intensity profile of light measured with respect to the optical fiber.

6. The apparatus of claim 3 wherein the quantitative parameter is an angular alignment of the optical fiber.

7. The apparatus of claim 2 wherein the medical device is a combination OCT and IVUS probe and the data collection subsystem comprises an ultrasound transducer and an optical element.

8. The apparatus of claim 7 wherein the quantitative parameter comprises one more measurements configured to specify an offset between the ultrasound transducer and the optical element.

9. The apparatus of claim 8 wherein the optical element is selected from the group consisting of the optical fiber, a GRIN lens, a beam director, a beam forming end face of the optical fiber and an angled surface in optical communication with an interferometer.

10. The apparatus of claim 2 wherein the medical device is an image data collection probe and the data collection subsystem comprises an ultrasound transducer and wherein the quantitative parameter is a physical characteristic of the ultrasound transducer.

11. The apparatus of claim 10 wherein the physical characteristic is an acoustic efficiency, one or more dimensions of the ultrasound transducer, and an ultrasound transducer drive voltage.

12. The apparatus of claim 3 further comprising a reader configured to receive the quantitative parameter from the data encoder.

13. The apparatus of claim 12 further comprising a control system comprising a processor configured to execute a software-based method using the quantitative parameter received by the reader, the control system in communication with the reader.

14. The apparatus of claim 13 wherein executing the software-based method using the quantitative parameter is faster or otherwise has a signal to noise ratio improvement with respect to a resultant image relative to executing the software-based method without having the quantitative parameter.

15. The apparatus of claim 14 wherein the software-based method changes an optical path length dimension used by the control system in response to optical path length data encoded as the quantitative parameter and measured with respect to the image data collection probe.

16. A method of collecting image data comprising:
receiving a quantitative parameter from a data encoder, the quantitative parameter derived from or comprising a measured value of a disposable data collection probe component;
compensating for variations in disposable data collection probes using the quantitative parameter as an input to a control system; and
collecting image data using the disposable data collection probe in response to one or more control signals generated at least partially based upon the quantitative parameter.

17. The method of claim 16 wherein the disposable data collection probe component is selected from the group consisting of an optical fiber, an ultrasound transducer, a GRIN lens, a beam director, a sheath, electrical leads, a catheter, and a flush solution.

18. The method of claim 16 wherein the step of compensating for variations further comprises the step of operating within an acoustic efficiency range at least partially provided by an acoustic efficiency encoded as the quantitative parameter.

19. The method of claim 16 wherein the step of compensating for variations further comprises the step of tracking relative positions of an acoustic beam and an optical beam using one or offset measurements encoded as the quantitative parameter.

20. The method of claim 16 further comprising the step of increasing a signal to noise ratio in an image formed using the image data by adjusting for a path imbalance due to a deviation from a predetermined optical fiber length and the optical fiber length measured with respect to the disposable data collection probe.

21. The method of claim 16 wherein the quantitative parameter is configured to identify a data collection probe.
